(19) United States
(12) Patent Application Publication
(10) Pub. No.: US 2025/0359880 A1
(43) Pub. Date: Nov. 27, 2025

(54) DEVICES AND METHODS TO CENTER PRESSURE WAVE GENERATORS WITHIN A SEGMENTED BALLOON

(71) Applicant: Cardiovascular Systems, Inc., Santa Clara, CA (US)

(72) Inventors: Gregory A. BOLDENOW, St. Michael, MN (US); Timothy B. PETRICK, Brooklyn Park, MN (US)

(21) Appl. No.: 19/212,312

(22) Filed: May 19, 2025

Related U.S. Application Data
(60) Provisional application No. 63/650,044, filed on May 21, 2024.

(51) Int. Cl. A61B 17/22; A61B 17/00; A61M 25/00; A61M 25/10

(52) U.S. Cl. CPC A61B 17/22022; A61M 25/0054; A61M 25/1011; A61B 2017/00557; A61B 2017/22025; A61B 2017/22055; A61B 2017/22065; A61M 2210/12

(57) ABSTRACT

A catheter and segmented balloon system for a lithotripsy system having improved accommodation for curved configurations, which minimizes or prevents damage to the balloon during generation of the lithotripsy shock wave (e.g., resulting from electrical arcing of electrode pairs or use of a laser). Embodiments of the catheter include an elongate member with alternating flexibility regions wherein stiffer regions are configured to support at least one lithotripsy emitter, and wherein more flexible regions are disposed on either side of each stiffer region. This configuration minimizes risk that the section with the lithotripsy emitter(s), will tend to bend in a manner that positions the emitter close to the balloon wall. Embodiments of the segmented balloon system include balloon segments with interposed sections between each of the segmented balloons wherein the outer diameter of an inflated balloon segment is larger than the outer diameter of the interposed section.

### CROSS-REFERENCE TO RELATED APPLICATIONS

This application claims the benefit of and priority to U.S. Provisional Patent Application Ser. No. 63/650,044 filed May 21, 2024, and entitled “CENTER POSITIONING SEGMENTED FULID-FILLABLE ENCLOSURE AND ELONGATE MEMBER WITH ALTERNATING FLEXIBLITY REGIONS FOR IVL SYSTEMS, DEVICES, AND METHODS,” which application is expressly incorporated herein by reference in its entirety.

### BACKGROUND

### 1. The Field of the Invention

The present disclosure relates to intravascular and other lithotripsy systems generally. More specifically the present disclosure relates to fluid-fillable enclosures and elongate members associated with the fluid-fillable enclosures of intravascular and other lithotripsy systems.

### 2. The Relevant Technology

A variety of techniques and instruments have been developed for use in the removal or repair of tissue in arteries and similar body passageways, including removal and/or cracking of calcified lesions within the passageway and/or formed within the wall defining the passageway. A frequent objective of such techniques and instruments is the removal of atherosclerotic plaque in a patient's arteries. Atherosclerosis is characterized by the buildup of fatty deposits (atheromas) in the intimal layer (i.e., under the endothelium) of a patient's blood vessels. Very often over time what initially is deposited as relatively soft, cholesterol-rich atheromatous material hardens into a calcified atherosclerotic plaque, often within the vessel wall. Such atheromas restrict the flow of blood, cause the vessel to be less compliant than normal, and therefore often are referred to as stenotic lesions or stenoses, the blocking material being referred to as stenotic material. If left untreated, such stenoses can cause angina, hypertension, myocardial infarction, strokes and the like.

Angioplasty, or balloon angioplasty, is an endovascular treatment procedure by widening narrowed or obstructed arteries or veins, typically to treat arterial atherosclerosis. A collapsed balloon is typically passed through a pre-positioned catheter and over a guide wire into the narrowed occlusion and then inflated to a fixed pressure.

The balloon forces expansion of the occlusion within the vessel and the surrounding muscular wall until the occlusion yields from the radial force applied by the expanding balloon, opening up the blood vessel with an inner diameter that is similar to the native vessel in the occlusion area and, thereby, improving blood flow. Generally, known IVL devices further include a voltage pulse generator in operative communication with one or more pairs of electrodes mounted on a catheter and within an inflatable balloon, which electrodes are used to generate an acoustic shock wave within the balloon, which shock wave is transmitted to the surrounding vessel to be treated. Such shock waves an aid in breaking up the calcified diseased tissue, to improve blood flow. Such IVL devices may exhibit greater efficacy than conventional ballon angioplasty, which may not include generation of any such shock wave.

Intravascular lithotripsy systems, devices and methods have been described by Applicant, e.g., See PCT/2022/074607, filed Aug. 5, 2022 and entitled “INTRAVASCULAR LITHOTRIPSY BALLOON SYSTEMS, DEVICES AND METHODS”, and PCT/US2023/085868, filed Dec. 23, 2023 and entitled “INTRAVASCULAR LITHOTRIPSY SYSTEM WITH IMPROVED DURABILITY, EFFICIENCY AND PRESSURE OUTPUT VARIABILITY”, the entire contents of each of which are hereby incorporated by reference in their entirety.

As illustrated in FIG. 1, a diagrammatic or schematic layout of portions of an exemplary and known IVL system 12 is provided. The illustrative IVL system 12 comprises a catheter assembly 14 including an elongate body, embodied as a catheter having guidewire 15, and a fluid-filled member 16 configured to contain conductive fluid therein, exemplified by an inflatable balloon, disposed near one end of the body and arranged to receive fluid for inflation to facilitate IVL therapy. A set of dischargeable spaced-apart electrodes or other lithotripsy emitter 18 are shown arranged within the exemplary balloon 16, where electrodes 18 are spaced apart by a gap 17 from each other to create a spark or electrical arc between the set of spaced-apart electrodes 18.

The IVL system embodiments described herein may be used in connection with electrodes that are within a fluid-filled member 16 configured to contain a fluid, e.g., a conductive fluid, therein. The fluid-filled member 16 may include an inflatable balloon as shown in FIG. 1, which may be compliant or non-compliant and serves to contain the fluid such that the spaced-apart electrodes 18 are preferably fully submerged within the contained fluid. In addition, the fluid-filled member 16 may comprise a fillable member that is at least partially rigid and/or not flexible. In other embodiments, the fluid-filled member 16 may contain the fluid therein and wherein the set of spaced-apart electrodes or other emitter 18 are most typically fully submerged within the contained fluid.

Alternatively, the IVL system control embodiments of the present disclosure may be used in connection with electrodes that are not located or surrounded by a fluid-filled or fillable member 16. In these embodiments, the IVL system may comprise one or more sets of spaced-apart electrodes or emitters 18 that may be continuously or periodically exposed to saline or other fluid and, during the exposure, the IVL system may generate an electrical arc between the spaced-apart electrodes or emitters 18. In all embodiments discussed herein, one or more sets of spaced-apart electrodes or emitters 18 may be provided. While electrode emitters may be generally described, other types of lithotripsy emitters are also possible (e.g., a laser).

Each of the spaced-apart electrodes in FIG. 1 is arranged in communication (as suggested by dashed line conductors) with an electric pulse generation system, or voltage pulse generator, 20 to receive high voltage electrical energy for spark generation to create pressure shock waves for IVL therapy. In the illustrative embodiment, one electrode may be grounded and the other provided with high voltage from the electric pulse generation system 20, although in some embodiments, any suitable voltage differential may be applied. The electric pulse generation system 20 includes an IVL control system 22 comprising a processor 24 configured for executing instructions stored on memory 26 and communications signals via circuitry 28 for IVL operations according to the processor governance. The processor 24, memory 26, and circuitry 28 are arranged in communication with each other (as suggested via dashed lines) to facilitate disclosed operations.

An exemplary known IVL system is shown schematically in FIG. 2, illustrating one method for applying voltage pulses to the system, resulting in current flow through the exemplary system to each set of spaced-apart electrodes 18, wherein the illustrated emitters 18 are connected in series. FIG. 2 illustrates two sets of spaced-apart electrodes or emitters 18 defined within each support body SB. Application of a voltage pulse of sufficient magnitude and/or duration from the electric pulse generation system 20 to the sets of serially connected spaced-apart electrodes 18 will result in a successive production of electrical arcs across spark gaps 17 in an order conforming with the connection method of the electrodes with the electric pulse generation system 20. In the illustrated case, the connection method is a series connection.

Targeted treatment regions such as calcified occlusions and/or lesions within blood vessels or other bodily conduits may occur within a curvilinear space. FIG. 3 illustrates a known IVL device 10 simulating placement within a curvilinear blood vessel or conduit. As shown, the inner electrode support member 13 curves within the balloon 14 to contact, or nearly contact, the balloon material on a portion of an outer radius of the curving inner electrode support member 13. This configuration places pairs of spaced-apart electrodes E in contact with, or in very close proximity to, the balloon material, positionally biasing the inner electrode support member 13 and spaced-apart electrode pairs E toward the side of the balloon 14. When the IVL system is activated to create electrical arcs, as discussed above, between the spaced-apart electrodes E, the resultant heat and energy from the generated electrical arcs can damage the balloon material, leading to unwanted weakening and, potentially, dangerous ruptures of the balloon 14. In this event, among other things, fluid in the balloon 14, contaminated with byproducts of the electrical arcing processes, can be released into the patient's circulatory system. Furthermore, such rupture is unacceptable, as it requires the practitioner to remove the ruptured balloon, for replacement with another balloon, to continue the treatment procedure. For a myriad of reasons, such rupture is unacceptable.

In order to reduce incidence of such rupture, it would be beneficial to provide a lithotripsy system that would minimize or eliminate, positional biasing of the electrodes or other lithotripsy emitters in very close proximity to the wall of the fluid-filled balloon when treating an occlusion or lesion within a curvilinear or curving vessel or conduit, for example a peripheral blood vessel. Various embodiments of the present disclosure address at least some such issues.

### BRIEF SUMMARY OF THE INVENTION

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

An embodiment of the present disclosure relates to a catheter for an intravascular or other lithotripsy (“IVL”) system. Such a catheter may include an elongate member including a fluid-inflatable balloon, with alternating regions of flexibility located along the elongate member within the fluid-inflatable balloon. The alternating regions of flexibility may include at least one region configured to support at least one lithotripsy emitter (e.g., a pair of electrodes, a laser, or other emitter), wherein the at least one region includes a flexibility that is less than the flexibility of the remaining alternating regions of flexibility.

Another embodiment is directed to a catheter for an intravascular or other lithotripsy (“IVL”) system including an elongate member having a distal region disposed along a distal portion of the elongate member, and one or more pairs of regions longitudinally spaced apart from each other and disposed along a portion of the elongate member that is proximal to the distal region, where each one of the one or more pairs of regions are longitudinally spaced apart from each other by an intermediate region. The distal region and the intermediate region are more flexible than the one or more pairs of regions.

Another embodiment is directed to a segmented balloon system for an intravascular or other lithotripsy (“IVL”) system, including an elongate member and two or more segmented balloons configured to surround a portion of the elongate member in a watertight association with the elongate member. Each of the two or more segmented balloons are spaced longitudinally from an adjacent segmented balloon or enclosure by an interposed section including an outer diameter when inflated. Each of the segmented balloons may include a tapered proximal section, a middle cylindrical section, and a tapered distal section. When the balloon is inflated, the middle section includes an outer diameter that is greater than the outer diameter of the interposed section.

Another embodiment is directed to a segmented balloon system for an intravascular or other lithotripsy (“IVL”) system, including an elongate member and an outer member including a lumen that is configured to receive the elongate member therein. Two or more segmented balloons are provided, configured to surround a portion of the elongate member in a watertight association with the elongate member at a distal end and with the outer member at a proximal end. Each of the two or more segmented balloons are spaced longitudinally from an adjacent segmented balloon or enclosure by an interposed section including an outer diameter when inflated, where each of the segmented balloons includes a tapered proximal section, a middle cylindrical section, and a tapered distal section. When inflated, the middle section includes an outer diameter that is greater than the outer diameter of the interposed section.

In any of the described embodiments, the at least one lithotripsy emitter may include at least a pair of spaced apart electrodes, or a laser.

In any of the described embodiments, the at least one region may be disposed along the elongate member at a location that is distal of, and adjacent to, an alternating region of flexibility that is greater than the flexibility of the at least one region. In addition, the at least one region may be disposed along the elongate member at a location that is proximal of, and adjacent to, an alternating region of flexibility that is greater than the flexibility of the at least one region. In other words, the at least one region may be between alternating regions of flexibility that are more flexible than the at least one region.

In any of the described embodiments, the at least one region may include 2, 3, 4, or 5 regions longitudinally spaced apart from each other by an alternating region of flexibility that is greater than the flexibility of the 2, 3, 4 or 5 regions.

In any of the described embodiments, the catheter may further include a fluid-inflatable balloon surrounding at least the at least 2, 3, 4 or 5 regions.

In any of the described embodiments, the fluid-inflatable balloon may be configured to surround at least part of the remaining alternating regions of flexibility.

In any of the described embodiments, the catheter may include one or more lithotripsy emitters disposed along the elongate member within each region of the one or more pairs of regions.

In any of the described embodiments, the catheter may include a fluid-inflatable balloon configured to surround the one or more pairs of regions and each intermediate region.

In any of the described embodiments, the fluid-inflatable balloon surrounds at least part of the one or more pairs of regions that are proximal to the distal region.

In any of the described embodiments of a segmented balloon system, two or more segmented balloons may be configured to be in fluid communication with each other.

In any of the described embodiments of a segmented balloon system, the elongate member may be engaged by, or in contact with, at least one interposed section when the segmented balloon system is curved or bent.

In any of the described embodiments of a segmented balloon system, the interposed section that is engaged with the elongate member may be configured to space the elongate member from an internal surface of the two or more segmented balloons.

In any of the described embodiments of a segmented balloon system, the system may include one or more pairs of spaced electrodes or other lithotripsy emitters associated with the elongate member and located within each one of the two or more segmented balloons.

In any of the described embodiments of a segmented balloon system may include any of the catheter systems described, with two or more segmented balloons configured to surround a portion of the elongate member in a watertight association with the elongate member, each of the two or more segmented balloons being spaced longitudinally from an adjacent segmented balloon or enclosure by an interposed section including an outer diameter when inflated. Each of the segmented balloons may include a tapered proximal section, a middle cylindrical section, and a tapered distal section. Once inflated, the middle section may have an outer diameter that is greater than the outer diameter of the interposed section.

In any of the described embodiments of a segmented balloon system, each one of the two or more segmented balloons may be configured to surround the at least one region configured to support at least one lithotripsy emitter.

In any of the described embodiments of a segmented balloon system, the elongate member may include a region of flexibility with a flexibility that is greater than the flexibility of the at least one region configured to support at least one lithotripsy emitter, wherein the region of flexibility is surrounded by the interposed section.

In any of the described embodiments of a segmented balloon system, the two or more segmented balloons may be configured to be in fluid communication with each other.

In any of the described embodiments of a segmented balloon system, the elongate member may be engaged by, or in contact with, at least one interposed section when the segmented balloon system is curved or bent.

In any of the described embodiments of a segmented balloon system, each interposed section that is engaged with the elongate member may be configured to space the elongate member from an internal surface of the two or more segmented balloons.

Related methods of use may include use of any of the described systems to perform an IVL or other lithotripsy treatment procedure.

The present devices differ from various earlier disclosed configurations. For example, US2024/0226512 discloses tapered balloon configurations, but does not include interposed sections between adjacent balloon segments.

CN113730768A discloses an expansion ballon and balloon expansion catheter where a limiting part or structure is provided external to the balloon body, to limit expansion of the radial dimension where the external limiting structure is positioned. In an embodiment of the presently described configurations, no external limiting structures are present, placed over the balloon body as in such reference.

US2022/0313359 discloses an intravascular lithotripsy balloon that includes a proximal region diameter, a distal region diameter, and transition region therebetween, where the transition region diameter varies. There is no provision of varying stiffness regions, or interposed sections between adjacent balloon segments, as in at least some of the presently disclosed embodiments.

U.S. Pat. No. 11,583,339 discloses an asymmetrical balloon for an intravascular lithotripsy device including an energy guide that receives energy and guides energy from the energy source into the balloon interior. The energy guide includes a guide distal end that is on the balloon's central axis when the balloon is inflated. The configuration provides the greatest distance between the generated plasma and the balloon wall, in an effort to reduce balloon rupture. There is no provision of a segmented balloon with interposed sections, or recognition of the problems that occur when the balloon curves around a bend within the vasculature, which tends to position the generated plasma closer to the balloon wall than when in a straight configuration.

U.S. Pat. No. 11,484,327 discloses an intravascular lithotripsy device that includes protective structure(s) (such as a cage) positioned on the exterior of the balloon, in an effort to reduce physical contact between the exterior of the balloon with the calcified plaque lesion appended to the vessel wall, to reduce risk of rupture or damage to the balloon wall. As noted above relative to CN113730768A, in an embodiment of the presently described configurations, no such external cage like structures are present, placed over the balloon body.

Each of the above references is herein incorporated by reference in its entirety. While the above references attempt to address various issues, they do not necessarily recognize or address problems associated with curvature, and the tendency of the inner member that supports the lithotripsy emitters to bend towards the balloon wall, when in a curved configuration, which issues are addressed by at least some embodiments of the present disclosure.

Additional features and advantages of exemplary implementations of the invention will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims or may be learned by the practice of such exemplary implementations as set forth hereinafter.

### BRIEF DESCRIPTION OF THE DRAWINGS

A description of various aspects and features of the invention will be rendered by reference to various representative embodiments thereof illustrated in the appended drawings. It is appreciated that these drawings depict only typical embodiments of the invention and are therefore not to be considered limiting of its scope.

FIG. 1 illustrates a schematic view of an IVL system.

FIG. 2 illustrates a schematic view of part of an IVL system.

FIG. 3 illustrates a side, cut-away view of a portion of a prior art IVL system.

FIG. 4 illustrates a side, cut-away view of an embodiment of the present disclosure.

FIG. 5 illustrates a side, cut-away view of another embodiment of the present disclosure.

FIG. 6 illustrates a side, cut-away view of another embodiment of the present disclosure.

FIG. 7 illustrates a side, cut-away view of another embodiment of the present disclosure.

FIG. 8 illustrates a side, cut-away view of another embodiment of the present disclosure.

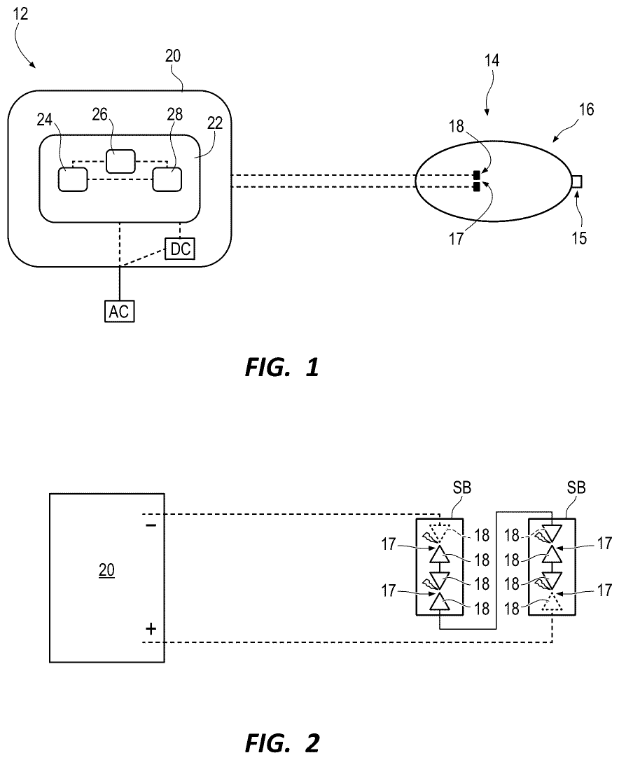

### DETAILED DESCRIPTION

One or more specific embodiments of the present disclosure will be described below. In an effort to provide a concise description of these embodiments, some features of an actual embodiment may be described in the specification. It should be appreciated that in the development of any such actual embodiment, as in any engineering or design project, numerous embodiment-specific decisions will be made to achieve the developers' specific goals, such as compliance with system-related and business-related constraints, which may vary from one embodiment to another. It should further be appreciated that such a development effort might be complex and time consuming, but would nevertheless be a routine undertaking of design, fabrication, and manufacture for those of ordinary skill having the benefit of this disclosure.

While the present disclosure will describe various particular implementations, it should be understood that the devices, systems, and method described herein may be applicable in other environments, and to other uses. Additionally, elements described in relation to any embodiment depicted and/or described herein may be combinable with elements described in relation to any other embodiment depicted and/or described herein.

FIG. 4 illustrates an embodiment of the present disclosure and comprises a lithotripsy segmented balloon system 100 having a series of segmented balloons B1, B2, B3, B4, an outer member 102 operatively associated in a watertight connection with a proximal cylindrical end section 101 of the balloon system 100, an inner member 103 extending through a lumen of the outer member 102 and further distally beyond a distal end of the outer member 102 through the interior space of the balloon system 100 to operatively engage a distal cylindrical end 110 of the segmented balloon system 100. While principally described as cylindrical, it will be appreciated that other shapes are also possible (e.g., oval cross-section or otherwise).

The various embodiments are described herein as comprising “balloons” or a series of balloon structures that are in fluid communication throughout. It is to be understood, however, that the term “balloon” is not limiting and is to be construed broadly. As a result, any suitable watertight enclosure structure is within the scope of the present disclosure.

The first segmented balloon B1 is shown as including the proximal cylindrical end section 101 operatively associated with an outer surface of the outer member 102 to provide a watertight engagement, a proximal tapered section 120 adjacent to the proximal cylindrical end section 101, a middle cylindrical section 122 adjacent to the proximal tapered section 120, and a distal tapered section 124 adjacent to the middle cylindrical section 122.

The second segmented balloon B2 is shown as including a proximal tapered section 128, a middle cylindrical section 130 adjacent to the proximal tapered section 128, and a distal tapered section 132 adjacent to the middle cylindrical section 130.

The third segmented balloon B3 is shown as including a proximal tapered section 136, a middle cylindrical section 138 adjacent to the proximal tapered section 136, and a distal tapered section 140 adjacent to the middle cylindrical section 138.

The fourth segmented balloon B4 is shown as including a proximal tapered section 144, a middle cylindrical section 146 adjacent to the proximal tapered section 144, a distal tapered section 148 adjacent to the middle cylindrical section 146, and a distal cylindrical section 110 adjacent to the distal tapered section 148 and which is operatively associated with an outer surface of the inner member 103 to provide a watertight engagement.

In the illustrated embodiment, the proximal cylindrical end section 101 surrounds a distal part of the outer member 102 and the distal cylindrical end section 110 surrounds a portion of the inner member 103. The illustrated system 100 comprises the inner member 103 disposed within, and extending distally from, the outer member 102. As a result, the outer diameter of the outer member 102 will be larger than the outer diameter of the inner member 103. Consequently, the outer diameter of the proximal cylindrical section 101 will, in the illustrated embodiment, be larger than the outer diameter of the distal cylindrical section 110. The skilled artisan will recognize that this arrangement or configuration is not limiting and that, for example, a single elongate member with a substantially constant outer diameter may be used to create the operative watertight association with the proximal cylindrical end section 101 and the distal cylindrical end section 110. In this embodiment, the outer diameters of the proximal cylindrical end section 101 and the distal cylindrical end section 110 may be substantially equal.

A first interposed cylindrical section 126 is shown provided between the distal tapered section 124 of the first segmented balloon B1 and the proximal tapered section 128 of the second segmented balloon B2. The outer diameter of the first interposed cylindrical section 126 is less than the outer diameter of the middle cylindrical sections 122, 130 of the first and second segmented balloons B1, B2.

A second interposed cylindrical section 134 is shown provided between the distal tapered section 132 of the second segmented balloon B2 and the proximal tapered section 136 of the third segmented balloon B3. The outer diameter of the second interposed cylindrical section 134 is less than the outer diameter of the middle cylindrical sections 130, 138 of the second and third segmented balloons B2, B3.

A third interposed cylindrical section 142 is provided between the distal tapered section 140 of the third segmented balloon B3 and the proximal tapered section 144 of the fourth segmented balloon B4. The outer diameter of the third interposed cylindrical section 142 is less than the outer diameter of the middle cylindrical sections 138, 146 of the third and fourth segmented balloons B3, B4.

The segmented balloons B1-B4 may be in fluid communication with each other, and with an external fluid reservoir (not shown but as will be familiar to the skilled artisan in light of the present disclosure) such that the balloon system 100 may be inflated with fluid, or deflated, as an overall system 100. In other embodiments, individual segmented balloons B1-B4 may be independently inflated to allow customized inflation/deflation and actuation of the spaced apart electrodes or other lithotripsy emitter(s) within the individual segmented balloons B1-B4 that are inflated.

In the illustrated embodiment, the middle cylindrical sections 122, 130, 138 and 146 may all comprise substantially equal outer diameters when inflated. In other embodiments, the outer diameters 122, 130, 138 and 146 may comprise outer diameters that are different from one another when inflated. In some embodiments, the outer diameters of the middle cylindrical sections 122, 130, 138, 146 may increase moving in the proximal direction such that the distal-most middle cylindrical section 146 may have the smallest outer diameter and the proximal-most middle cylindrical section 122 may have the largest outer diameter. An inverse arrangement is also possible (e.g., where balloon section 146 has the largest outer diameter, and balloon section 122 has the smallest outer diameter. Other configurations are also possible (e.g., balloon section 130 and/or 138 may have a larger diameter than the other balloon sections). All combinations of outer diameter for the middle cylindrical sections 122, 130, 138, 146 when inflated are within the scope of the present disclosure.

Similarly, the outer diameters of the interposed cylindrical sections 126, 134 and 142 may comprise outer diameters that are substantially the same when inflated, or may comprise outer diameters that are not the same when inflated.

In some embodiments, an axial length of the interposed cylindrical sections 126, 134 and 142 may be substantially equal. In other embodiments, the axial length of the interposed cylindrical sections may differ from one another. All combinations of the lengths of the interposed cylindrical sections are within the scope of the present disclosure.

Similarly, the axial length of the segmented balloons B1-B4 may be substantially equal, that is the distance between, in the case of segmented balloon B1, a proximal end of the proximal tapered section and a distal end of the distal tapered section. In other embodiments, the axial length of the segmented balloons B1-B4 may not be equal. Similarly, the middle cylindrical sections 122, 130, 138, 146 may be of substantially equal length. In other embodiments, the middle cylindrical sections 122, 130, 138, 146 may be of unequal length. For example, the distal-most middle cylindrical section 122 may have a length that is less than a length of the remaining middle cylindrical sections. All combinations of middle cylindrical section length are within the scope of the present disclosure.

FIG. 4 further illustrates the inner member 103 disposed through the segmented balloon system 100 along a longitudinal axis A when the balloon system 100 is straight as in FIG. 4. In addition, each balloon B1, B2, B3 and B4 is shown as including at least one pair of spaced-apart electrodes, respectively designated E1, E2, E3 and E4 as illustrated. It will be appreciated that alternative lithotripsy emitters are also possible and within the scope of the present disclosure. For example, a laser could be used to generate a plasma, similar to an electrode pair. For example, such a laser may be used to heat a metallic target, which upon heating generates a plasma, with an accompanying cavitation bubble, or the like. In either case, one or more lithotripsy emitters E1-E4 may be provided. The electrodes E1-E4 are illustratively located within the middle cylindrical sections 122,130, 138 and 146. However, the skilled artisan will recognize that the number of pairs of spaced-apart electrodes or other lithotripsy emitters within the balloon structures B1-B4 may be one, or may be more than one. In addition, the artisan will recognize that the location of the one or more pairs of electrodes or other lithotripsy emitters within each balloon structure B1-B4 can be within the respective middle cylindrical section, the respective proximal tapered section, and/or the respective distal tapered section. All such combinations are within the scope of the present disclosure.

One of the benefits of the smaller outer diameters of the interposed cylindrical sections 126, 134 and 142, relative to the larger outer diameters of the middle cylindrical sections 122, 130, 138 and 146, is that the distance between the inner member 103 and the balloon material within the interposed cylindrical sections 126, 134 and 142 is less than the distance between the inner member 103 and the balloon material within the middle cylindrical sections 122, 130, 138 and 146. This differential allows for control of a radial position of the inner member 103 and electrodes E1-E4 during curvature of the system 100.

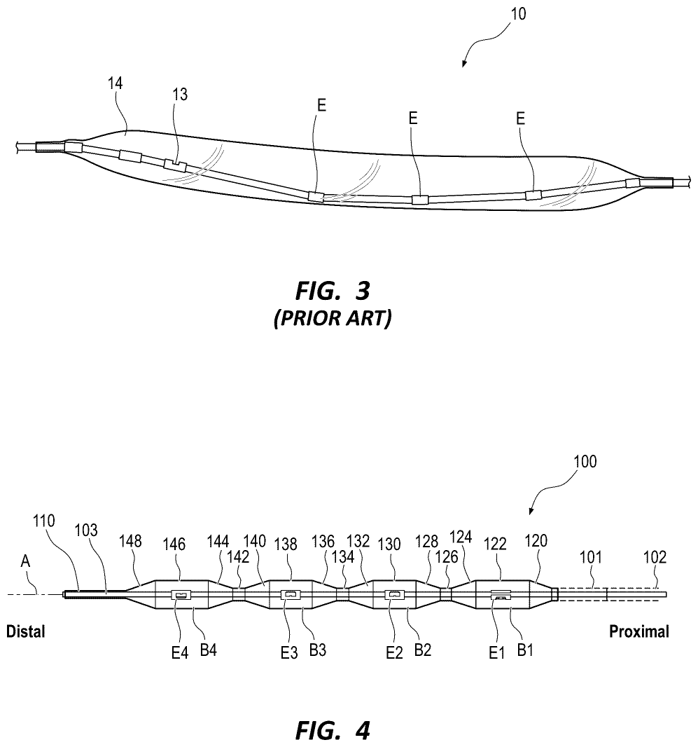

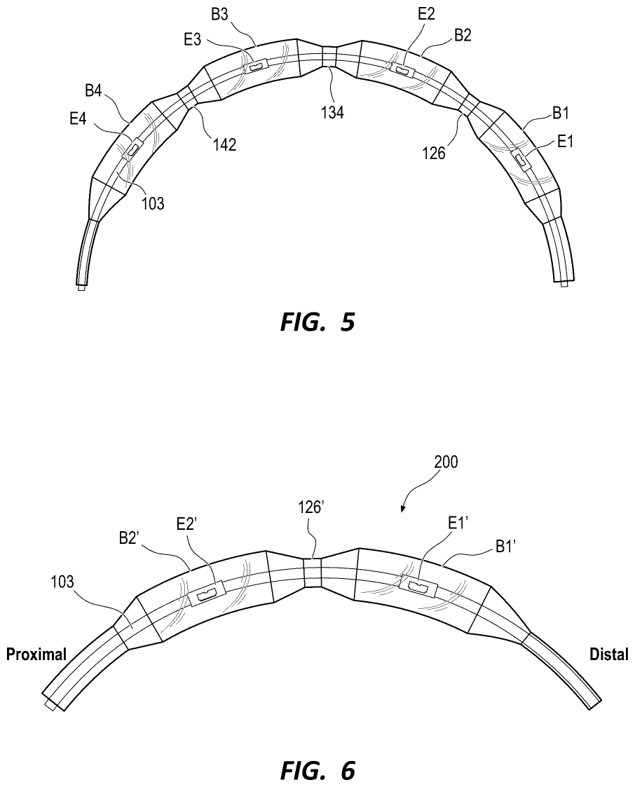

As shown in FIG. 5, the segmented balloon system 100 of FIG. 4 is now illustrated as curved to simulate a curved vessel or conduit. Referring back to FIG. 3, this curvature in the prior art device results in undesirable positional bias of the inner support member 13 relative to the balloon 14, such that individual electrode pairs or other emitters E in FIG. 3 are positioned undesirably close to the surface of balloon 14.

In the embodiment of FIG. 5, in contrast, the interposed cylindrical sections 126, 134 and 142 provide, by virtue of the smaller outer diameter relative to the larger outer diameters of the middle cylindrical sections 122, 130, 138 and 146, a barrier or a physical limit to the radial extension of the inner member 103 and provides a surface against which the inner member 103 is pressed such that additional curvature of the inner member 103 occurs. In an embodiment, the inside diameter associated with interposed sections 126, 134 and 142 may be similar to the outer diameter of inner member 103, to maximize such control. For example, any gap between the inner member 103 and the inner diameter of interposed sections 126, 134 and/or 142 may be less than 100%, less than 75%, less than 50%, less than 25%, less than 20% or less than 10% relative to the outer diameter of the inner member 103, or relative to the inner diameter of the interposed section.

In some curvatures, a portion of the inner member 103 may contact one or more of the interposed cylindrical sections 126. However, the inner member 103 is prevented from moving further radially toward the material (e.g., balloon material) of the segmented balloons B1, B2, B3 and B4 and is, instead, urged into a tighter, more radiused, curvature than if the interposed cylindrical sections 126, 134, 142 were not present. Thus, the interposed cylindrical sections 126, 134 and 142 function to space the electrode pair(s) from the balloon material and prevent the one or more electrode pairs or other lithotripsy emitters E1-E4 from damaging the segmented balloons B1-B4 during electrical arcing or other generation of the acoustic shock wave when the segmented balloon system 100 is in a curved configuration by preventing or limiting positional bias of the inner member 103, and the associated electrode pairs or other lithotripsy emitters E1-E4, relative to the balloon material.

As the skilled artisan will appreciate, the segmented balloon system 100 of FIGS. 4 and 5 is not limited to the illustrated balloon system with four segments. The system as described in connection with FIGS. 4 and 5 can be readily implemented with a segmented balloon system comprising two or more segmented balloons (e.g., 2, 3, 4, 5, 6 or more segments). For example, as shown in FIG. 6, a segmented balloon system 200 with two segmented balloons, a proximal segmented balloon B1′ and a distal segmented balloon B2′, separated by a single interposed cylindrical section 126′, with inner member 103 disposed through the system 200 and supporting at least one electrode or other lithotripsy emitter E1′, E2′ within each of the segmented balloons B1′ and B2′, wherein the structures, features and functions of the elements denoted with a prime symbol after the number are the same as the corresponding numbered elements described in connection with FIGS. 4 and 5. Thus, a segmented balloon system of the above-described embodiments may comprise at least two segmented balloons and at least one interposed cylindrical section.

In some embodiments, the interposed cylindrical section(s) may comprise a stiffer balloon material than the adjacent segmented balloons to further help prevent the inner member 103, and associated electrodes or other lithotripsy emitters, from biasing to one side of the segmented balloons when in a curved configuration. For example, the interposed sections may comprise a higher durometer material (e.g., having a higher Shore A durometer hardness) than the adjacent balloon segments. In an embodiment, they may comprise a rigid material (e.g., rather than an elastomeric material).

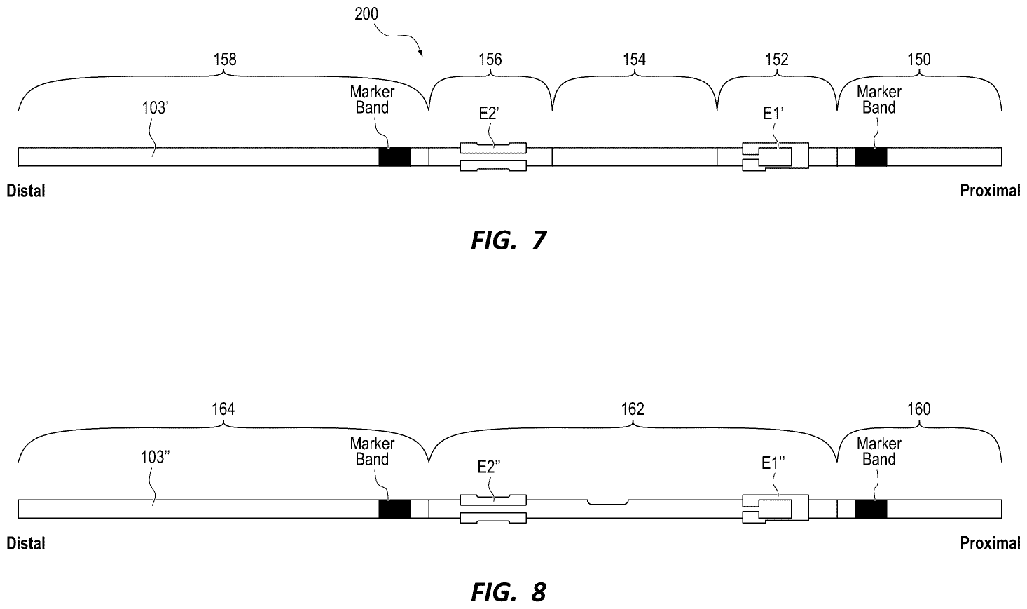

Turning now to FIGS. 7 and 8, exemplary illustrations of particular embodiments of the inner member 103 are provided. The segmented balloon systems discussed above function to prevent or minimize positional bias of the inner member 103 toward a side of one or more of the segmented balloons when in a curved configuration.

FIGS. 7 and 8 provide additional positional bias prevention mechanisms embodied within the structure of the inner member 103, using regions of relatively increased and decreased flexibility within the segmented balloon(s). FIGS. 7 and 8 illustrate one non-limiting example of an inner member 103 comprising two electrode pair structures (or other lithotripsy emitters). The principles discussed below are at least applicable to inner members 103 comprising two or more electrode pairs or other lithotripsy emitters that are spaced longitudinally from each other and/or one or more balloons, including a balloon such as that illustrated in FIG. 3, or including two more segmented balloon structures as disclosed herein.

FIG. 7 provides an exemplary inner member 103′ that may be used to improve a balloon such as that shown in FIG. 3, or in combination with any of the exemplary segmented balloon embodiments shown in FIGS. 4-6. Thus, moving from proximal to distal along the inner member 103′, a first region comprising a relatively flexible material 150 is provided and formed of, e.g., a polyether block amide (PEBAX) thermoplastic elastomer material. A marker band is located within the first region of relatively flexible material 150. Distally adjacent with the first region of relatively flexible material 150, is a second region comprising stiffer material 152 (relative to the first region 150) that may comprise polyimide or other similar material that is stiffer and less flexible than the material of the first region 150. Distally adjacent to the second region 152 is a third region (e.g., an intermediate region) of relatively more flexible, less stiff material 154 which may comprise the same material as that of the first region 150. Distally adjacent to the third region is a fourth region of relatively stiffer and less flexible material 156 and which may comprise the same material as that of the second region 152. Distally adjacent to the fourth region 156 is a fifth region of relatively more flexible, less stiff material 158 that may comprise the same material as that of the first region 150 and/or the third (e.g., intermediate) region 154. The materials of more stiff regions 152 and 156 may have a Shore A durometer value that is greater than that of the less stiff, more flexible regions 150, 154, and 158. In another embodiment, the materials of more stiff regions 152 and 156 may have a Young's modulus value that is greater than that of the less stiff, more flexible regions of 150, 154, and 158.

A first electrode or other lithotripsy emitter E1′ is shown as located on the inner member 103′ within the second, stiffer region 152. A second electrode or other lithotripsy emitter E2′ is shown as located on the inner member 103′ within the fourth, stiffer region 156. Interposed between the two electrodes or other lithotripsy emitters E1′ and E2′ is the third region of relatively more flexible, less stiff material. Where the lithotripsy emitter comprises a laser, only a single lithotripsy emitter may be present (e.g., whereas where the lithotripsy emitters are electrodes, there will typically be at least two spaced apart electrodes, in order to generate a plasma arc therebetween).

Accordingly, in the two segmented balloon embodiment of FIG. 6, mounting or locating the electrodes or other lithotripsy emitters E1′ and E2′ within the second region 152 and the fourth region 156, respectively, assists in removing positional bias of the inner member 103 in the regions 152, 156 where the electrodes E1′ and E2′ are located when the inner member is in a curved position by making it harder to flex the second and fourth regions 152, 156. As a result, the electrodes E1′ and E2′ are limited in movement in a radial direction and are therefore better held or spaced away from the surface of the balloon.

Generally, the radius of the curving inner member 103 and the segmented balloon structures discussed above may achieve a curving configuration wherein the radii of the inner member 103 and the segmented balloon structure surrounding the inner member 103 are substantially the same. This is in contrast to the known device of FIG. 3, wherein the balloon's radius is typically greater than that of the inner member 13, causing the inner member 12, and related electrode pairs E, to bow radially outwardly toward the balloon material.

FIG. 8 illustrates another embodiment of an inner member 103″ that may be used to improve a system comprising a prior art balloon such as that of FIG. 3, or used with an embodiment including a segmented balloon system as described herein, to minimize and/or prevent positional bias of the inner member and associated electrodes relative to the surrounding balloon surface when in a curved configuration. Accordingly, moving from the proximal to the distal direction along the inner member 103″, a first region of increased flexibility 160 is provided and which may comprise a PEBAX material. Distally adjacent to the first region 160 is a second region of relatively less flexible material with increased stiffness 162, relative to the first region 160. Distally adjacent to the second region 162 is a third region of increased flexibility 164 that may comprise the same or a similarly flexible material as the first region 160.

The inner member 103″ is shown as including at least two electrode pairs E1″ and E2″ (or another lithotripsy emitter) located along the inner member 103″ within the second region 162 of increased stiffness.

Similar to the embodiment of FIG. 7, placing the electrode pairs E1″ and E2″ or other lithotripsy emitter(s) in the stiffer second region 162 assists in removing positional bias of the inner member 103 in the second region 162 when the inner member is in a curved position by making it more difficult to flex the second region 162. As a result, the electrodes E1″ and E2″ (or other lithotripsy emitter in this region) are limited in movement in a radial direction and better held away from the surface of the balloon.

Because the second region 162 holds both pairs of electrodes E1″ and E2″ (or includes whatever other type of lithotripsy emitter may be used), a single balloon may be used, e.g., any of a wide variety of IVL balloons as one of skill in the art will understand in light of the present disclosure. Alternatively, a 2-segmented balloon system may also be used as described above, wherein each electrode E1″ and E2″ (or other lithotripsy emitter) is located within its own adjacently located segmented balloon with an interposed cylindrical section as described above in, e.g., any of FIGS. 4-6.

The inner members 103′ and 103″ of FIGS. 7 and 8 may be implemented alone, e.g., with any desired lithotripsy balloon, or in combination with embodiments of the segmented balloons described herein, to minimize and/or prevent positional bias of the inner member and associated electrodes relative to the surrounding balloon surface when in a curved configuration. Better spacing the electrodes away from the balloon surface makes it less likely that the balloon surface will be damaged by the generated plasma arc. When other lithotripsy emitters configured to generate a shock wave are used (e.g., a laser), a similar benefit is achieved, as the plasma and accompanying shock wave generated by a laser can damage the balloon surface in a similar manner than damage may be caused by plasma arcing and associated shock wave generated by a pair of spaced apart electrodes.

As above for the embodiments described in conjunction with FIG. 7, the materials of more stiff region 162 may have a Shore A durometer value that is greater than that of the less stiff, more flexible regions 160, 164. In another embodiment, the materials of more stiff region 162 may have a Young's modulus value that is greater than that of the less stiff, more flexible regions of 160, 164.

The elongate members 103′ and 103″ of FIGS. 7 and 8 may comprise at least the following non-limiting examples 1 and 2.

Example 1 (e.g., as shown in FIG. 7): A catheter for intravascular or other lithotripsy (“IVL”) system, comprising:
an elongate member comprising:
a distal region disposed along a distal portion of the elongate member, and
one or more pairs of regions longitudinally spaced apart from each other and disposed along a portion of the elongate member that is proximal to the distal region, wherein each one of the two or more pairs of regions are longitudinally spaced apart from each other by an intermediate region,
wherein the distal region and the intermediate region are more flexible than the one or more pairs of regions.

Example 2 (e.g., as shown in FIG. 8): A catheter for intravascular or other lithotripsy (“IVL”) system, comprising:
an elongate member comprising a fluid-inflatable balloon, and
alternating regions of flexibility located along the elongate member within the fluid-inflatable balloon,
wherein the alternating regions of flexibility comprise at least one region configured to support at least one pair of spaced-apart electrodes or another lithotripsy emitter, wherein the at least one region comprises a flexibility that is less than the flexibility of the remaining alternating regions of flexibility.

In addition, the elongate member 103′ and 103″ embodiments may be combined with embodiments including a segmented balloon described herein, e.g., as follows in the below non-limiting exemplary example 3.

Example 3: A segmented balloon system for an intravascular or other lithotripsy (“IVL”) system, comprising:
the catheter of Example 1 or 2;
two or more segmented balloons configured to surround a portion of the elongate member in a watertight association with the elongate member, each of the two or more segmented balloons being spaced longitudinally from an adjacent segmented balloon or enclosure by an interposed section comprising an outer diameter when inflated,
wherein each of the segmented balloons comprises a tapered proximal section, a middle cylindrical section comprising an outer diameter when inflated that is greater than the outer diameter of the interposed section when inflated, and a tapered distal section.

Non-limiting segmented balloon embodiments may also include non-limiting exemplary examples 4 and 5:

Example 4: A segmented balloon system for an intravascular or other lithotripsy (“IVL”) system, comprising:
an elongate member;
two or more segmented balloons configured to surround a portion of the elongate member in a watertight association with the elongate member, each of the two or more segmented balloons being spaced longitudinally from an adjacent segmented balloon or enclosure by an interposed section comprising an outer diameter when inflated,
wherein each of the segmented balloons comprises a tapered proximal section, a middle cylindrical section comprising an outer diameter when inflated that is greater than the outer diameter of the interposed section when inflated, and a tapered distal section.

Example 5: A segmented balloon system for an intravascular or other lithotripsy (“IVL”) system, comprising:
an elongate member;
an outer member comprising a lumen and configured to receive the elongate member therein;
two or more segmented balloons configured to surround a portion of the elongate member in a watertight association with the elongate member at a distal end and with the outer member at a proximal end, each of the two or more segmented balloons being spaced longitudinally from an adjacent segmented balloon or enclosure by an interposed section comprising an outer diameter when inflated;
wherein each of the segmented balloons comprises a tapered proximal section, a middle cylindrical section comprising an outer diameter when inflated that is greater than the outer diameter of the interposed section when inflated, and a tapered distal section.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

Any ranges disclosed herein also encompass any and all overlap, sub-ranges, and combinations thereof. Additional ranges may be defined within any values provided herein, and are within the scope of the present disclosure. Language such as “up to,” “at least,” “greater than,” “less than,” “between,” and the like includes the number(s) recited.

Following are some further example embodiments of the invention. These are presented only by way of example and are not intended to limit the scope of the invention in any way. Further, any example embodiment can be combined with one or more of the example embodiments.

Embodiment 1. A catheter for an intravascular or other lithotripsy (“IVL”) system, comprising:
an elongate member comprising a fluid-inflatable balloon; and
alternating regions of flexibility located along the elongate member within the fluid-inflatable balloon;
wherein the alternating regions of flexibility comprise at least one region configured to support at least one lithotripsy emitter, wherein the at least one region comprises a flexibility that is less than the flexibility of the remaining alternating regions of flexibility.

Embodiment 2. The catheter of embodiment 1, wherein the at least one lithotripsy emitter comprises at least a pair of spaced apart electrodes, or a laser.

Embodiment 3. The catheter of embodiment 1, wherein the at least one region is disposed along the elongate member at a location that is distal of, and adjacent to, an alternating region of flexibility that is greater than the flexibility of the at least one region, and wherein the at least one region is disposed along the elongate member at a location that is proximal of, and adjacent to, an alternating region of flexibility that is greater than the flexibility of the at least one region.

Embodiment 4. The catheter of embodiment 3, wherein the at least one region comprises two regions longitudinally spaced apart from each other by an alternating region of flexibility that is greater than the flexibility of the two regions.

Embodiment 5. The catheter of embodiment 3, wherein the at least one region comprises three regions, each of the three regions being longitudinally spaced apart by an alternating region of flexibility that is greater than the flexibility of the three regions.

Embodiment 6. The catheter of embodiment 3, wherein the at least one region comprises four regions, each of the four regions being longitudinally spaced apart by an alternating region of flexibility that is greater than the flexibility of the four regions.

Embodiment 7. The catheter of embodiment 3, wherein the at least one region comprises five regions, each of the five regions being longitudinally spaced apart by an alternating region of flexibility that is greater than the flexibility of the five regions.

Embodiment 8. The catheter of embodiment 4, wherein the fluid-inflatable balloon surrounds at least the two regions.

Embodiment 9. The catheter of embodiment 5, wherein the fluid-inflatable balloon surrounds at least the three regions.

Embodiment 10. The catheter of embodiment 6, wherein the fluid-inflatable balloon surrounds at least the four regions.

Embodiment 11. The catheter of embodiment 7, wherein the fluid-inflatable balloon surrounds at least the five regions.

Embodiment 12. The catheter of embodiment 8, wherein the fluid-inflatable balloon is configured to surround at least part of the remaining alternating regions of flexibility.

Embodiment 13. A catheter for an intravascular or other lithotripsy (“IVL”) system, comprising:
an elongate member comprising:
a distal region disposed along a distal portion of the elongate member, and
one or more pairs of regions longitudinally spaced apart from each other and disposed along a portion of the elongate member that is proximal to the distal region, wherein each one of the one or more pairs of regions are longitudinally spaced apart from each other by an intermediate region;
wherein the distal region and the intermediate region are more flexible than the one or more pairs of regions.

Embodiment 14. The catheter of embodiment 13, further comprising one or more lithotripsy emitters disposed along the elongate member within each region of the one or more pairs of regions.

Embodiment 15. The catheter of embodiment 14, wherein the one or more lithotripsy emitters comprise one or more pairs of spaced-apart electrodes, or a laser.

Embodiment 16. The catheter of embodiment 13, further comprising a fluid-inflatable balloon configured to surround the one or more pairs of regions and each intermediate region.

Embodiment 17. The catheter of embodiment 16, wherein the fluid-inflatable balloon surrounds at least part of the one or more pairs of regions that are proximal to the distal region.

Embodiment 18. A segmented balloon system for an intravascular or other lithotripsy (“IVL”) system, comprising:
an elongate member; and
two or more segmented balloons configured to surround a portion of the elongate member in a watertight association with the elongate member, each of the two or more segmented balloons being spaced longitudinally from an adjacent segmented balloon or enclosure by an interposed section comprising an outer diameter when inflated;
wherein each of the segmented balloons comprises a tapered proximal section, a middle cylindrical section comprising an outer diameter when inflated that is greater than the outer diameter of the interposed section when inflated, and a tapered distal section.

Embodiment 19. The segmented balloon system of embodiment 18, wherein the two or more segmented balloons are configured to be in fluid communication with each other.

Embodiment 20. The segmented balloon system of embodiment 19, wherein the elongate member is engaged by, or in contact with, at least one interposed section when the segmented balloon system is curved or bent.

Embodiment 21. The segmented balloon system of embodiment 20, wherein each interposed section that is engaged with the elongate member is configured to space the elongate member from an internal surface of the two or more segmented balloons.

Embodiment 22. The segmented balloon system of embodiment 21, further comprising (i) one or more pairs of spaced electrodes or (ii) other lithotripsy emitter(s) associated with the elongate member and located within each one of the two or more segmented balloons.

Embodiment 23. A segmented balloon system for an intravascular or other lithotripsy (“IVL”) system, comprising:
the catheter of any of embodiments 1-17; and
two or more segmented balloons configured to surround a portion of the elongate member in a watertight association with the elongate member, each of the two or more segmented balloons being spaced longitudinally from an adjacent segmented balloon or enclosure by an interposed section comprising an outer diameter when inflated;
wherein each of the segmented balloons comprises a tapered proximal section, a middle cylindrical section comprising an outer diameter when inflated that is greater than the outer diameter of the interposed section when inflated, and a tapered distal section.

Embodiment 24. The segmented balloon system of embodiment 23, wherein each one of the two or more segmented balloons are configured to surround the at least one region configured to support at least one lithotripsy emitter.

Embodiment 25. The segmented balloon system of embodiment 24, wherein the elongate member further comprises a region of flexibility with a flexibility that is greater than the flexibility of the at least one region configured to support at least one lithotripsy emitter, wherein the region of flexibility is surrounded by the interposed section.

Embodiment 26. A segmented balloon system for an intravascular or other lithotripsy (“IVL”) system, comprising:
an elongate member;
an outer member comprising a lumen and configured to receive the elongate member therein; and
two or more segmented balloons configured to surround a portion of the elongate member in a watertight association with the elongate member at a distal end and with the outer member at a proximal end, each of the two or more segmented balloons being spaced longitudinally from an adjacent segmented balloon or enclosure by an interposed section comprising an outer diameter when inflated;
wherein each of the segmented balloons comprises a tapered proximal section, a middle cylindrical section comprising an outer diameter when inflated that is greater than the outer diameter of the interposed section when inflated, and a tapered distal section.

Embodiment 27. The segmented balloon system of embodiment 26, wherein the two or more segmented balloons are configured to be in fluid communication with each other.

Embodiment 28. The segmented balloon system of embodiment 26, wherein the elongate member is engaged by, or in contact with, at least one interposed section when the segmented balloon system is curved or bent.

Embodiment 29. The segmented balloon system of embodiment 28, wherein each interposed section that is engaged with the elongate member is configured to space the elongate member from an internal surface of the two or more segmented balloons.

Embodiment 30. A method of treating an intravascular lesion using the catheter of one or more of embodiments 1-17.

Embodiment 31. A method of treating an intravascular lesion using the segmented balloon system of one or more of embodiments 18-29.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A catheter for an intravascular or other lithotripsy (“IVL”) system, comprising:
an elongate member comprising a fluid-inflatable balloon; and
alternating regions of flexibility located along the elongate member within the fluid-inflatable balloon;
wherein the alternating regions of flexibility comprise at least one region configured to support at least one lithotripsy emitter, wherein the at least one region comprises a flexibility that is less than the flexibility of the remaining alternating regions of flexibility.

2. The catheter of claim 1, wherein the at least one lithotripsy emitter comprises at least a pair of spaced apart electrodes, or a laser.

3. The catheter of claim 1, wherein the at least one region is disposed along the elongate member at a location that is distal of, and adjacent to, an alternating region of flexibility that is greater than the flexibility of the at least one region, and wherein the at least one region is disposed along the elongate member at a location that is proximal of, and adjacent to, an alternating region of flexibility that is greater than the flexibility of the at least one region.

4. The catheter of claim 3, wherein the at least one region comprises two regions longitudinally spaced apart from each other by an alternating region of flexibility that is greater than the flexibility of the two regions.

5. The catheter of claim 3, wherein the at least one region comprises three regions, each of the three regions being longitudinally spaced apart by an alternating region of flexibility that is greater than the flexibility of the three regions.

6. The catheter of claim 3, wherein the at least one region comprises four regions, each of the four regions being longitudinally spaced apart by an alternating region of flexibility that is greater than the flexibility of the four regions.

7. The catheter of claim 3, wherein the at least one region comprises five regions, each of the five regions being longitudinally spaced apart by an alternating region of flexibility that is greater than the flexibility of the five regions.

8. The catheter of claim 4, wherein the fluid-inflatable balloon surrounds at least the two regions.

9. The catheter of claim 5, wherein the fluid-inflatable balloon surrounds at least the three regions.

10. (canceled)

11. (canceled)

12. The catheter of claim 8, wherein the fluid-inflatable balloon is configured to surround at least part of the remaining alternating regions of flexibility.

13. A catheter for an intravascular or other lithotripsy (“IVL”) system, comprising:
an elongate member comprising:
a distal region disposed along a distal portion of the elongate member, and
one or more pairs of regions longitudinally spaced apart from each other and disposed along a portion of the elongate member that is proximal to the distal region, wherein each one of the one or more pairs of regions are longitudinally spaced apart from each other by an intermediate region;
wherein the distal region and the intermediate region are more flexible than the one or more pairs of regions.

14. The catheter of claim 13, further comprising one or more lithotripsy emitters disposed along the elongate member within each region of the one or more pairs of regions.

15. The catheter of claim 14, wherein the one or more lithotripsy emitters comprise one or more pairs of spaced-apart electrodes, or a laser.

16. The catheter of claim 13, further comprising a fluid-inflatable balloon configured to surround the one or more pairs of regions and each intermediate region.

17. The catheter of claim 16, wherein the fluid-inflatable balloon surrounds at least part of the one or more pairs of regions that are proximal to the distal region.

18. A segmented balloon system for an intravascular or other lithotripsy (“IVL”) system, comprising:
an elongate member; and
two or more segmented balloons configured to surround a portion of the elongate member in a watertight association with the elongate member, each of the two or more segmented balloons being spaced longitudinally from an adjacent segmented balloon or enclosure by an interposed section comprising an outer diameter when inflated;
wherein each of the segmented balloons comprises a tapered proximal section, a middle cylindrical section comprising an outer diameter when inflated that is greater than the outer diameter of the interposed section when inflated, and a tapered distal section.

19. The segmented balloon system of claim 18, wherein the two or more segmented balloons are configured to be in fluid communication with each other.

20. The segmented balloon system of claim 19, wherein the elongate member is engaged by, or in contact with, at least one interposed section when the segmented balloon system is curved or bent.

21. The segmented balloon system of claim 20, wherein each interposed section that is engaged with the elongate member is configured to space the elongate member from an internal surface of the two or more segmented balloons.

22. The segmented balloon system of claim 21, further comprising (i) one or more pairs of spaced electrodes or (ii) other lithotripsy emitter(s) associated with the elongate member and located within each one of the two or more segmented balloons.

23-31. (canceled)
